(19) United States
(12) Patent Application Publication
(10) Pub. No.: US 2019/0142881 A1
(43) Pub. Date: May 16, 2019

(54) BACTERIOPHAGE COMPOSITIONS AND USES THEREOF

(71) Applicant: YALE UNIVERSITY, New Haven, CT (US)

(72) Inventors: Paul TURNER, New Haven, CT (US); Benjamin CHAN, Salt Lake City, UT (US); John E. WERTZ, Cheshire, CT (US)

(21) Appl. No.: 16/095,041

(22) PCT Filed: Apr. 25, 2017
(86) PCT No.: PCT/US17/29317

§ 371 Date: Oct. 19, 2018

Related U.S. Application Data
(60) Provisional application No. 62/327,208, filed on Apr. 25, 2016.

(51) Int. Cl. A61K 35/76; C12N 7/00; A61K 45/06; A61P 31/04

(52) U.S. Cl. CPC A61K 35/76; C12N 7/00; A61K 45/06; C12N 2795/10132; C12N 2795/10121; C12N 2795/10131; A61P 31/04

(57) ABSTRACT

The present invention includes compositions and methods of bacteriophage to increase antibiotic sensitivity in bacteria. In one aspect, the invention includes a method of increasing antibiotic sensitivity in multi-drug resistant (MDR) bacteria. Another aspect includes a pharmaceutical composition comprising a lytic bacteriophage. Yet another aspect includes a method of treating a multi-drug resistant bacterial infection in a subject. Yet another aspect includes a method of disrupting a pathogenic bacteria associated with a biofilm and compositions for use thereof.

### CROSS-REFERENCE TO RELATED APPLICATION

This application claims priority to U.S. Provisional Application Ser. No. 62/327,208, filed Apr. 25, 2016, the content of which is incorporated by reference herein in its entirety.

### STATEMENT OF RIGHTS TO INVENTIONS MADE UNDER FEDERALLY SPONSORED RESEARCH

This invention was made with government support under 1051093 awarded by the National Science Foundation. The government has certain rights in the invention.

### BACKGROUND OF THE INVENTION

Widespread and inappropriate uses of chemical antibiotics have selected for multi-drug resistant (MDR) bacterial pathogens, presenting more frequently in human infections and contributing significantly to morbidity. Some bacteria even show evolved resistance to ‘drugs of last resort’, resulting in emergent strains that are pan-drug-resistant (PDR). One example is the Gram-negative bacterium, Pseudomonas aeruginosa, a prevalent opportunistic MDR pathogen that is poised to become a common PDR disease problem. Humans readily encounter P. aeruginosa, which thrives in both natural and artificial environments, varying from lakes and estuaries to hospitals and household sink drains. P. aeruginosa causes biofilm-mediated infections, including catheter associated urinary tract infections, ventilator associated pneumonia, and infections related to mechanical heart valves, stents, grafts and sutures (Cole, S. J., et al., Infection and Immunity 82, 2048-2058 (2014)). Individuals with cystic fibrosis, severe burns, surgical wounds and/or compromised immunity are particularly at risk for P. aeruginosa infections, especially acquired in hospitals.

P. aeruginosa is a ubiquitous Gram-negative, rod-shaped bacterium prevalent in natural and artificial environments (Remold, S. K., et al. Microb Ecol. 62(3), 505-17 (2011)). Adaptation to different habitats has allowed P. aeruginosa to persist in many human-associated environments, most notably in hospitals, where it is increasingly associated with nosocomial infections (Emori, T. G., et al., Clin Microbiol Rev. 6(4), 428-42 (1993)). These infections are difficult to manage, in part due to intrinsic antibiotic resistance resulting from decreased membrane permeability, active antibiotic efflux, and other chromosomally encoded enzymes. Further complicating the problem of P. aeruginosa infections are their ability to form biofilms, herein referred to as “P. aeruginosa biofilms” or “Pseudomonas aeruginosa biofilms”. Biofilm-mediated infections are notoriously difficult to manage, having seemingly much higher resistance to chemical antimicrobials (Stewart, P. S., et al., Lancet 358(9276), 135-08 (2001)) and often form following sub-lethal concentrations of antibiotics (Hoffman, L. R., et al., Nature 436(7054), 1171-5 (2005)). This elevated resistance may be due to exopolymeric substances in the biofilm matrix that slow diffusion of antibiotics and reduce effective concentrations. Furthermore, slow-growing cells present in the biofilm (e.g., persister cells) may have sufficiently reduced metabolisms to withstand bacteriostatic antibiotics that target metabolically active bacteria (Lewis, K. Biochemistry (Mosc). 70(2),267-74 (2005)). As a result, biofilms may also act as a reservoir for the dissemination of infections throughout the body which could greatly prolong infection duration and severity. Prosthetic vascular graft infections are of significant concern due to the elevated mortality and morbidity rates. A common culprit, P. aeruginosa, presents a serious challenge due to its intrinsic antibiotic resistance and ability to form biofilms on prosthetic material.

Prosthetic vascular graft infections are catastrophic events which present serious challenges to surgeons and place heavy economic burdens on patients and the healthcare system. The reported incidence can vary from 0.6% to 9.5% depending on the site of the vascular graft (Kieffer, E., et al., J Vasc Surg. 33(4), 671-8 (2001); Schild, A. F., et al., J Vasc Access 9(4), 231-5 (2008)). There are currently no clear algorithms for the management of prosthetic vascular graft infections. The basic principles, however, involve systemic antibiotics, debridement of infected tissue, partial or complete graft excision, and secondary revascularization (Bunt, T. J. Cardiovasc Surg. 9(3), 225-33 (2001)). However, many patients presenting with vascular graft infections have significant comorbidities and are often critically ill, making surgical management even more difficult and in certain cases ill-advised. Despite best management, mortality and morbidity rates remain high with conservative estimates for both over 20% (O'Connor, et al., S. J Vasc Surg. 44(1), 38-45 (2006); Perera, G. B., et al., Vasc Endovascular Surg. 40(1), 1-10 (2006)). Reinfection rates are also significant after initial treatment, highlighting the inadequacy of current treatment modalities at eradicating the infecting organism. As the number of procedures involving vascular grafts continues to rise with an aging population and increasing prevalence of atherosclerosis and diabetes, new strategies are sorely needed.

P. aeruginosa infections are notoriously difficult to manage due to low antibiotic permeability of the outer membrane and mechanisms of antibiotic resistance that allow cross resistance to multiple classes and types of antibiotics. Clinically significant levels of antibiotic resistance are mostly caused by interplay between the efficient outer membrane (OM) permeability barrier, ubiquitous periplasmic β-lactamases, and multi-drug resistance (MDR) efflux pumps. These pumps have broad substrate specificity and may act synergistically with the permeability barrier to result in significant intrinsic resistance to many antimicrobials. These pumps expel the antimicrobial from the cell into the surrounding space, and the antimicrobials then have to pass through the OM permeability barrier to regain entry to the cell. Thus, the MDR pumps can effect significant resistance even when their transporter activity is quite low, as long as the OM functions as an effective barrier.

Synergy between efflux and the permeability barrier is necessary for effective drug resistance. Efflux pumps are transport proteins that are found in both Gram-positive and -negative bacteria, as well as in eukaryotic organisms. Pumps may be specific for one substrate or may transport a range of structurally dissimilar compounds (including antibiotics of multiple classes); such pumps can be associated with multi-drug resistance (MDR). Efflux pumps can also impact iron uptake, bile tolerance, quorum sensing, and other host colonization factors.

In the bacteria domain, there are five major families of efflux transporter: MF (major facilitator), MATE (multidrug and toxic efflux), RND (resistance-nodulation-division), SMR (small multidrug resistance) and ABC (ATP binding cassette). All these systems utilize the proton motive force as an energy source, apart from the ABC family, which utilizes ATP hydrolysis to drive the export of substrates. Transporters that efflux multiple substrates, including antibiotics, did not arise in response to the stresses of the antibiotic era. All bacterial genomes studied contain several different efflux pumps; this indicates their ancestral origins. It has been estimated that ˜5-10% of all bacterial genes are involved in transport and a large proportion of these encode efflux pumps.

Therefore, a need exists in the art to develop alternative methods for the management of antibiotic efflux of MDR in bacteria, like P. aeruginosa.

### SUMMARY OF THE INVENTION

The invention includes a method of increasing antibiotic sensitivity in pathogenic bacteria. The method comprises contacting the bacteria with a lytic bacteriophage, wherein the bacteriophage binds a molecule of an efflux pump in the bacteria and the bacteria either genetically resists bacteriophage infection or becomes infected and lysed by the bacteriophage, and wherein genetically resistant bacteria have impaired efflux pumps and increased sensitivity to antibiotics.

In some embodiments, the bacteria are contacted with bacteriophage at a multiplicity of infection (MOI) of bacteriophage to bacteria in the range of about 0.05 to about 50. In other embodiments, the bacteriophage binds a protein of a Mex efflux pump. In yet additional embodiments, the Mex protein is surface exposed protein. In some embodiments, the Mex protein is selected from the group consisting of OprM, MexA, MexB, MexX, and MexY. Further embodiments comprise contacting the genetically resistant bacteria with an antibiotic. In yet other embodiments, the pathogenic bacteria are multi-drug resistant (MDR) bacteria.

The invention additionally includes a pharmaceutical composition comprising a lytic bacteriophage, wherein the bacteriophage binds a molecule of an efflux pump on multi-drug resistant (MDR) bacteria. In some embodiments, the composition further comprises an antibiotic. In other embodiments, the composition further comprises one or more antibiotics.

Also included is a method of treating a multi-drug resistant bacterial infection in a subject in need thereof. The method comprises administering the pharmaceutical composition of the invention to the subject with the bacterial infection.

Also included is a method of disrupting a pathogenic bacteria associated with a biofilm. The method comprises contacting the bacteria with a lytic bacteriophage, wherein the bacteriophage binds a molecule of an efflux pump in the bacteria and the bacteria either genetically resists bacteriophage infection or becomes infected and lysed by the bacteriophage, and wherein genetically resistant bacteria have impaired efflux pumps and increased sensitivity to one or more antibiotics; and contacting the genetically resistant bacteria so identified with the one or more antibiotics, thereby disrupting the pathogenic bacteria associated with the biofilm.

In some embodiments, the pharmaceutical composition of the invention is administered directly to a site of the bacterial infection. Further embodiments comprise administering an antibiotic to the subject. In some embodiments, the antibiotic is administered before or after or is co-administered with the pharmaceutical composition.

In some embodiments, the bacteriophage is OMKO1. In other embodiments, the pathogenic bacteria is associated with a biofilm. In some embodiments, the pathogenic bacteria is Pseudomonas aeruginosa. In other embodiments, the Pseudomonas aeruginosa is a Pseudomonas aeruginosa biofilm.

In some embodiments, the bacteria is associated with a biofilm. In yet additional embodiments, the bacteria is Pseudomonas aeruginosa. In some embodiments, the Pseudomonas aeruginosa is a Pseudomonas aeruginosa biofilm.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a series of panels of tables and graphs illustrating that selection for phage resistance causes a trade-off resulting in significantly reduced Minimum Inhibitory Concentrations (MIC) to four drugs drawn from different antibiotic classes. LEFT: Average MIC±SD of four antibiotics for phage sensitive MDR bacteria (left column) and for spontaneous mutants of these bacteria resistant to phage OMKO1 (right column). RIGHT: Fold improvement of MIC for isolated strains resistant to OMKO1 (* p<0.05, ** p<0.01). For comparison, data for fold-increased sensitivity of transposon knockout PAO1-ΔoprM (phage resistant) is displayed as a vertical black line.

FIG. 2 is a graph and a panel of images illustrating that phage OMKO1 selects against the expression of OprM and, consequently, the function of the mexAB/XY-OprM efflux systems. Average cell densities (OD₆₀₀) of PA01-ΔmexR and PA01-ΔoprM over time in the presence of tetracycline (TET) (10 mg/L) and phage OMKO1 (green and red lines). PAO1 ΔmexR (blue, green) overexpresses mex-OprM and readily grows in TET to high densities alone due to active efflux of TET (blue) but is susceptible to phage infection (green). PAO1 ΔoprM grows poorly in the presence of TET (red) but is resistant to phage OMKO1 (yellow).

FIG. 3 is a schematic and two images illustrating that a phage increases MDR P. aeruginosa sensitivity to antibiotics by forcing a genetic trade-off. Bacteria are either sensitive to the phage (and less sensitive to antibiotics), left, or resistant to the phage (and more sensitive to antibiotics), right.

FIG. 4A is a schematic illustrating that: therapeutic concentrations of antibiotics are unable to penetrate biofilms due to poor permeability and depressed metabolism of biofilm constituents; Phage OMKO1 is able to replicate within bacteria present in biofilm; biofilm instability follows progression of infection by phage OMKO1 and as it replicates, maintenance of the biofilm decreases; and, with the biofilm disrupted, therapeutic concentrations of antibiotic are able to reach the target sites. Bacteria surviving phage OMKO1 infection are more susceptible to effluxed antibiotics.

FIG. 4B is a graph illustrating 24-hour growth of bacteria from 72-hour-old biofilms on Dacron sections exposed to decreasing Multiplicity of Infection (MOI) of phage OMKO1. The black horizontal line represents growth below the automated and visual limit of detection.

FIG. 4C is a graph illustrating 24-hour growth of bacteria from 72-hour-old biofilms on Dacron sections exposed to either ciprofloxacin or ceftazidime with and without phage OMKO1.

FIG. 5 is a a series of graphs illustrating antibiotic minimum inhibitory concentration (MIC) to four individual antibiotics by P. aeruginosa susceptible to phage OMKO1, left (black), and resistant to phage OMKO1, right (white). Reversal of clinical resistance in three antibiotics in which efflux is a major resistance mechanism was observed.

FIG. 6 is a graph illustrating regrowth of bacteria from 72-hour biofilms grown on different materials following treatment with phage OMKO1 (grey), phage OMKO1 & ceftazidime (2×MIC, black and grey), phage OMKO1 & ciprofloxacin (2×MIC, white and grey), ceftazidime alone at 2×MIC (black), ciprofloxacin alone at 2×MIC (white), and a control (growth medium only, dark grey) as measured by OD600 on an automated spectrophotometer.

FIG. 7 is an intraoperative photograph showing aortic graft and P. aeruginosa infection over myocardium.

FIG. 8 is a plot illustrating efficiency of plating (EOP) of phage OMKO1 isolated from lung and spleen tissue approximately 30 hours post-treatment in a mouse model of acute pneumonia. Black bar is average +/− standard deviation.

### DETAILED DESCRIPTION OF THE INVENTION

As more alternative therapies are considered to combat the rise of antibiotic resistant biofilm-associated infections, the use of bacteriophages, as described herein, presents a novel strategy to manage these difficult to manage infections.

The present invention includes compositions and methods of bacteriophage to increase antibiotic sensitivity in bacteria. In one aspect, the invention includes method of increasing antibiotic sensitivity in multi-drug resistant (MDR) bacteria. Another aspect includes a pharmaceutical composition comprising a lytic bacteriophage. Yet another aspect includes a method of treating a multi-drug resistant bacterial infection in a subject.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

As used herein, the terms “antibacterial activity” and “antimicrobial activity” with reference to a bacteriophage, isolated bacteriophage protein (or variant, derivative or fragment thereof), or bacteriophage product, are used interchangeably to refer to the ability to kill and/or inhibit the growth or reproduction of a microorganism, in particular, the bacteria of the species or strain that the bacteriophage infects. In certain embodiments, antibacterial or antimicrobial activity is assessed by culturing bacteria: gram-positive bacteria (e.g., S. aureus), gram-negative bacteria (e.g., K. pneumoniae, A. baumannii, E. coli, and P. aeruginosa) or bacteria not classified as either gram-positive or gram-negative, according to standard techniques (e.g., in liquid culture, on agar plates), contacting the culture with a bacteriophage or bacteriophage product and monitoring cell growth after the contact. For example, in a liquid culture, the bacteria may be grown to an optical density (“OD”) representative of a mid-point in exponential growth of the culture; the culture is exposed to one or more concentrations of one or more bacteriophage or bacteriophage product, and the OD is monitored relative to a control culture. Decreased OD relative to a control culture is representative of a bacteriophage or bacteriophage product exhibiting antibacterial activity (e.g., exhibits lytic killing activity). Similarly, bacterial colonies can be allowed to form on an agar plate, the plate exposed to a bacteriophage or bacteriophage product, and subsequent growth of the colonies evaluated related to control plates. Decreased size of colonies, or decreased total numbers of colonies, indicate a bacteriophage product.

By “attenuated” is meant the bacterium has a decreased virulence with respect to a wild-type bacterium. In particular, a bacterium has an attenuated virulence of about 10, 20, 30, 40, 50, 60, 70, 80% or more decrease in virulence as compared to a wild-type bacterium.

As used herein the terms “bacteriophage,” “lytic bacteriophage” and “bacteriophage products” refer to polypeptides, or fragments, variants or derivatives thereof, isolated from a bacteriophage of the invention, which polypeptide, or fragment, variant or derivative thereof, exhibits a biological function or activity associated with the bacteriophage from which it was isolated or derived (e.g., antimicrobial or antibacterial activity (e.g., lytic cell killing)).

By “effective amount” is meant the amount required to reduce or improve at least one symptom of a respiratory disorder, condition or disease relative to an untreated patient. The effective amount of airway epithelial cells used for therapeutic treatment of the respiratory disorder, condition or disease varies depending upon the manner of the specific disorder, condition or disease, extent of the disorder, condition or disease, and administration of the cells, as well as the age, body weight, and general health of the subject.

The term “efflux pump” refers to an active, protein transporter localized in the cell membrane that exports substrate(s). In bacteria, five classes of efflux pumps exist: MF (major facilitator), MATE (multidrug and toxic efflux), RND (resistance-nodulation-division), SMR (small multidrug resistance) and ABC (ATP binding cassette).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

A “vector” is a composition of matter that comprises a gene and that may be used to deliver the gene to the interior of a cell. Vector refers to any plasmid containing the gene that is capable of moving foreign sequences into the genomes of a target organism or cell.

As used herein, the term “fragment” as applied to a nucleic acid, is less than the whole.

By “host” or “host cell” is meant a cell, such as a mammalian cell, that harbors a pathogen, such as a bacterium. The pathogen can infect the host cell.

By “immune response” is meant the actions taken by a host to defend itself from pathogens or abnormalities. The immune response includes innate (natural) immune responses and adaptive (acquired) immune responses. Innate responses are antigen non-specific. Adaptive immune responses are antigen specific. An immune response in an organism provides protection to the organism against bacterial infections when compared with an otherwise identical subject to which the composition or cells were not administered or to the human prior to such administration.

By “infection” is meant a colonization of the host. Infection of a host can occur by entry through a membrane of the host, such as a phage passing through the cell membrane of a bacterium.

The term “bacterial infection” means the invasion of the host organism, animal or plant, by pathogenic bacteria. This includes the excessive growth of bacteria which are normally present in or on the body of the organism, but more generally, a bacterial infection is any situation in which the presence of a bacterial population(s) is damaging to a host organism. Thus, for example, an organism suffers from a bacterial infection when excessive numbers of a bacterial population are present in or on the organism's body, or when the effects of the presence of a bacterial population(s) is damaging to the cells, tissue, or organs of the organism.

By “infectious disease” is meant a disease or condition in a subject caused by a pathogen that is capable of being transmitted or communicated to a non-infected subject. Non-limiting examples of infectious diseases include bacterial infections, viral infections, fungal infections, and the like.

The term “isolated” refers to a material or an organism, such as bacteria, that is free to varying degrees from components or other organisms that normally accompany it as found in its native state. Isolated denotes a degree of separation from an original source or surroundings. An isolated bacterium is sufficiently free of other bacteria such that any contaminants do not materially affect growth, pathogencity, infection, etc. or cause other adverse consequences. That is, bacteria are isolated if they are substantially free of bacteria or materials. Purity and homogeneity are typically determined using analytical techniques, for example, single cell culturing. The term “purified” can denote that a cell gives rise to essentially one population.

By “multi-drug resistant,” “multi-drug resistance” or “MDR” is meant antimicrobial resistance to the effects of antibiotics or other antimicrobial drugs.

By “non-pathogenic” is meant an inability to cause disease.

By “pathogen” is meant an infectious agent, such as bacteria, capable of causing infection, producing toxins, and/or causing disease in a host.

By “disrupt” is meant to kill bacteria and/or to inhibit, slow, stop, or prevent bacterial replication and/or growth.

By “associated with a biofilm” is meant that the pathogen is present in and/or on a biofilm or forms a biofilm.

A “portion” of a polynucleotide means at least about twenty sequential nucleotide residues of the polynucleotide. It is understood that a portion of a polynucleotide may include every nucleotide residue of the polynucleotide.

“Proliferation” is used herein to refer to the reproduction or multiplication of similar forms, especially of bacteria. That is, proliferation encompasses production of a greater number of bacteria, and can be measured by, among other things, simply counting the numbers of bacteria, measuring incorporation of ³H-thymidine into the bacteria, and the like.

As used herein, “sample” or “biological sample” refers to anything, which may contain the cells of interest (e.g., cancer or tumor cells thereof) for which the screening method or treatment is desired. The sample may be a biological sample, such as a biological fluid or a biological tissue. In one embodiment, a biological sample is a tissue sample including pulmonary arterial endothelial cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

The term “strain” means bacteria or bacteriophage having a particular genetic content. The genetic content includes genomic content as well as recombinant vectors. Thus, for example, two otherwise identical bacterial cells would represent different strains if each contained a vector, e.g., a plasmid, with different phage open reading frame inserts.

A “subject” as used herein, may be a human or non-human organism. Non-human organisms include, but are not limited to, livestock, pets, aquaculture organisms, cultivated plants and crops. Preferably, the subject is human.

As used herein, the terms “treat,” treating,” “treatment,” and the like refer to reducing or improving an infectious disease or condition and/or one or more symptoms associated therewith. It will be appreciated that, although not precluded, treating an infectious disease or condition and/or one or more symptoms associated therewith does not require that the disorder, condition, disease or symptoms associated therewith be completely ameliorated or eliminated.

In the context of treating a bacterial infection a “therapeutically effective amount” or “pharmaceutically effective amount” indicates an amount of a composition comprising bacteriophage which has a therapeutic effect. This generally refers to the lysis of bacterial cells or, to some extent, of the acquisition of resistance (genetic evolution) of bacterial cells to bacteriophage infection.

By “virulence” is meant a degree of pathogenicity of a given pathogen or the ability of an organism to cause disease in another organism. Virulence refers to an ability to invade a host organism, cause disease, evade an immune response, and produce toxins.

By “bacterial virulence” is meant a degree of pathogenicity of bacteria. Bacterial virulence includes causing infection or disease in a host, producing agents that cause or enhance disease in a host, producing agents that cause or enhance disease spread to another host, and causing infection or disease in another host.

By “virulent” or “pathogenic” is meant a capability of a bacterium to cause a severe disease.

By “wildtype” is meant a non-mutated version of a gene, allele, genotype, polypeptide, or phenotype, or a fragment of any of these. It may occur in nature or produced recombinantly.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Description

“Phage therapy”, the application of lytic bacteriophages (or “phages”; viruses of bacteria) for the bio-control of bacteria, is one method for treating multi-drug-resistant (MDR) bacterial infections: the use of lytic (virulent) bacteriophages (bacteria-specific viruses) as self-amplifying ‘drugs’ that specifically target and kill bacteria. Lytic phages bind to one or more specific proteins on the surfaces of particular bacterial hosts, an intimacy that led to development of phage therapy as a biocontrol strategy which predated use of broad-spectrum chemical antibiotics. Due to the recent precipitous rise in antibiotic resistance, phage therapy has seen revitalized interest among Western physicians, buoyed by successful clinical trials demonstrating safety and efficacy.

However, one limitation of phage therapy is the abundant evidence that bacteria readily evolve resistance to phage infection. While multiple mechanisms of phage resistance exist, phage attachment to a receptor binding-site exerts selection pressure for bacteria to alter or down-regulate expression of the receptor, thereby escaping phage infection. Given the certainty of evolved phage-resistance, modern approaches to phage therapy must acknowledge and capitalize on this inevitability. Genetic trade-offs are often observed in biology, where organisms evolve one trait that improves fitness (a relative advantage in reproduction or survival), while simultaneously suffering reduced performance in another trait.

Described herein is an evolutionary-based strategy that forces a genetic trade-off: utilize phages that drive MDR bacterial pathogens to evolve increased phage resistance thereby increasing sensitivity to chemical antibiotics. Thus, this approach to phage therapy should be doubly effective; success is achieved when phage lyse the target bacterium, and success is also achieved when bacteria evolve phage resistance because they suffer increased sensitivity to antibiotics.

### Antibiotic Resistance

Many strains of bacteria have become antibiotic resistant, and some have become resistant to multiple antibiotics and chemotherapeutic agents, the phenomenon of multi-drug resistance. Some strains have become resistant to practically all of the commonly available agents. For example, methicillin-resistant Staphylococcus aureus (MRSA) is resistant to not only methicillin (which was developed to fight against penicillinase-producing S. aureus) but also aminoglycosides, macrolides, tetracycline, chloramphenicol, and lincosamides. Such strains are also resistant to disinfectants, and MRSA can act as a major source of hospital-acquired infections. An old antibiotic, vancomycin, was resurrected for treatment of MRSA infections. However, transferable resistance to vancomycin is now quite common in Enterococcus and found its way finally to MRSA.

The emergence of “pan-resistant” gram-negative strains, notably those belonging to P. aeruginosa and A. baumanii, occurred more recently, after most major pharmaceutical companies stopped the development of new antibacterial agents. Hence, there are almost no agents that could be used against these strains, in which an outer membrane barrier of low permeability and an array of efficient efflux pumps are combined with multitudes of specific resistance mechanisms.

Efflux pumps belonging to the resistance-nodulation-division (RND) family of transporters are the major multi-drug efflux (Mex) mechanism in both E. coli and P. aeruginosa. The pumps in this family consist of three components that function via active transport to move numerous molecules, including antibiotics, out of the cell: an antiporter that functions as a transporter (e.g., MexB, Mex D, MexF, MexY), an outer membrane protein that forms a surface-exposed channel (e.g., OprC, OprB, OprG, OprD, OprI, OprH, OprP, OprO, OprM, OprJ, OprN), and a periplasmic membrane fusion protein that links the two proteins (e.g., MexA, MexC, MexE, MexH, MexX). This system is the major efflux pump associated with intrinsic resistance among 17 possible RND efflux pumps in P. aeruginosa. P. aeruginosa is more resistant than E. coli due to a highly impermeable OM and the presence of multiple efflux systems. Inactivation of the Mex efflux pump renders P. aeruginosa more vulnerable to antibiotics than the average E. coli strain.

### Compositions

In one aspect, the invention includes a composition comprising a lytic bacteriophage, wherein the bacteriophage binds a molecule of an efflux pump on pathogenic bacteria, drug resistant bacteria, multi-drug resistant (MDR) bacteria, and/or pan-drug resistant (PDR) bacteria.

In one embodiment, the bacteriophage binds a protein, such as a surface exposed protein, of a Mex efflux pump. In another embodiment, the Mex protein is selected from the group consisting of OprM, MexA, MexB, MexX, and MexY.

In yet another embodiment, the composition further comprises an antibiotic. The antibiotic includes any commonly available agent, such as an antibiotic selected from, but not limited to, amoxicillin, erythromycin, penicillin, ciprofloxacin, azithromycin, ceftolozane/taxobactam, ceftazidime/acibactiam, tetracycline, imipenem/carbapenem, and any combination thereof.

The present invention also includes a pharmaceutical composition comprising the bacteriophage described herein. Pharmaceutical compositions comprise the bacteriophage in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); Magnetic Resonance and Computerized Tomography contrast agents; and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one aspect, the invention includes a composition or a pharmaceutical composition comprising the bacteriophage described herein, wherein the bacteriophage is OMKO1.

In another aspect, the invention includes a composition or a pharmaceutical composition comprising the bacteriophage described herein, wherein the pathogenic bacteria is associated with a biofilm.

In some embodiments, pathogenic bacteria is Pseudomonas aeruginosa. In some embodiments, the Pseudomonas aeruginosa is a Pseudomonas aeruginosa biofilm.

In another aspect, the invention includes a composition or a pharmaceutical composition comprising the bacteriophage described herein, wherein the bacteriophage disrupts the multi-drug resistant (MDR) bacteria, Pseudomonas aeruginosa.

In yet another aspect, the invention includes a composition or a pharmaceutical composition comprising the bacteriophage described herein, wherein the bacteriophage disrupts a Pseudomonas aeruginosa biofilm.

In some embodiments, the Pseudomonas aeruginosa biofilm is on a prosthetic material, e.g., Dacron, Gore-Tex, felt, and/or polypropylene, or any surgically relevant material.

In some embodiments, the bacteriophage is OMKO1.

### Methods

In another aspect, the invention includes a method of increasing antibiotic sensitivity in pathogenic bacteria. In some embodiments, the pathogenic bacteria are multi-drug resistant (MDR) bacteria. In some embodiments, the pathogenic bacteria are pan-drug resistant (PDR) bacteria.

The method comprises contacting the pathogenic bacteria with a lytic bacteriophage, wherein the bacteriophage binds a molecule of an efflux pump in the bacteria and the bacteria either genetically resists bacteriophage infection or becomes infected and lysed by the bacteriophage, and wherein genetically resistant bacteria have impaired efflux pumps and increased sensitivity to antibiotics.

In one embodiment, the bacteria are contacted with bacteriophage at a multiplicity of infection (MOI) of bacteriophage to bacteria in the range of about 0.0001 to about 10¹⁰. The MOI may range from about 0.0002 to about 10⁹, from about 0.0003 to about 10⁸, from about 0.0004 to about 10⁷, from about 0.0005 to about 10⁶, from about 0.0006 to about 10⁵, from about 0.0007 to about 10,000, from about 0.0008 to about 5,000, from about 0.0009 to about 2,500, from about 0.001 to about 1,000, from about 0.005 to about 500, from about 0.01 to about 100, from about 0.05 to about 50, from about 0.1 to about 10, or any range therebetween.

In another embodiment, the method further comprises contacting the genetically resistant bacteria with an antibiotic. The antibiotic includes any of the antibiotics described herein, any commonly known agent, and any combination thereof.

In another embodiment, the method comprises or further comprises contacting the genetically resistant bacteria with one or more antibiotics, e.g., 1-100 antibiotics or 1, 2, 3, 4, 5, 6, 7, 8, 9, 10, 11, 12, 13, 14, 15, 16, 17, 18, 19, 20, 21, 22, 23, 24, 25, or more antibiotics.

In yet another aspect, the invention includes a method of treating a multi-drug resistant bacterial infection in a subject in need thereof. The method comprises administering the pharmaceutical composition as described herein to the subject with the bacterial infection. In one embodiment, the composition is administered directly to a site of the bacterial infection. In another embodiment, the method further comprises administering an antibiotic as described herein to the subject. In one such embodiment, the antibiotic is co-administered with the pharmaceutical composition. In another such embodiment, the antibiotic is administered before or after the pharmaceutical composition is administered.

In some embodiments, the antibiotic can be administered minutes, hours, days, or weeks, before or after the pharmaceutical composition is administered, e.g.: 1, 5, 10, 15, 20, 30, or 45 minutes; 1, 2, 3, 4, 5, 6, 7, 8, 9, 10, 12, 14, 16, 18, 20, or 22 hours; 1, 2, 3, 4, 5, or 6 days; or 1 or 2 weeks, or any amount of time there between.

In another aspect, the invention includes a method of disrupting a pathogenic bacteria associated with a biofilm and compositions for use thereof.

In some embodiments, the biofilm is on Dacron and/or any other prosthetic material.

In some embodiments, the pathogenic bacteria is associated with a biofilm. In some embodiments, the pathogenic bacteria is Pseudomonas aeruginosa. In some embodiments, the Pseudomonas aeruginosa is a Pseudomonas aeruginosa biofilm.

### Administration/Dosing

In the clinical settings, delivery systems for a composition comprising a bacteriophage, wherein the bacteriophage binds a molecule of an efflux pump on multi-drug resistant (MDR) bacteria, can be administered to a subject by any of a number of methods, each of which is familiar in the art. For instance, a pharmaceutical formulation of the composition can be administered by inhalation, topically, locally or systemically, e.g., by intravenous injection, intramuscular injection, intraperitoneal injection, retro- or peribulbar injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the present invention to a subject, such as a mammal, for example a human, may be carried out using known procedures, at dosages and for periods of time effective to treat a disease or condition in the subject. An effective amount of the composition necessary to achieve a therapeutic effect may vary according to factors such as the extent of implantation; the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation.

Actual dosage levels of the cells in the pharmaceutical formulations of this invention may be varied so as to obtain an amount of the composition that are effective to achieve the desired therapeutic response for a particular subject, composition, and mode of administration, without being toxic to the subject.

### Routes of Administration

Routes of administration of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, topical, intra-orbital, intra-aural, intra-articular, and topical administration.

Suitable formulation of the compositions and dosages include, for example, dispersions, suspensions, solutions, beads, pellets, magmas, creams, pastes, plasters, lotions, discs, suppositories, liquid sprays for nasal, ocular or oral administration, aerosolized formulations for inhalation, compositions and formulations for intravesical administration and the like.

It should be understood that the formulations and compositions that would be useful in the present invention are not limited to the particular formulations set forth in the examples. The following examples are put forth so as to provide those of ordinary skill in the art with a complete disclosure and description of how to make and use the cells, differentiation methods, engineered tissues, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### Experimental Examples

Presented herein are in vitro and in vivo studies examining: lytic bacteriophages (phages) and their ability to disrupt pathogenic bacteria, e.g., Pseudomonas aeruginosa, and/or to disrupt biofilms on prosthetic materials; and the application of phages in the treatment of a chronic bacterial (P. aeruginosa) infection. The present invention includes compositions and pharmaceutical compostions of the phages and methods of their use in the disruption of P. aeruginosa and/or P. aeruginosa biofilms.

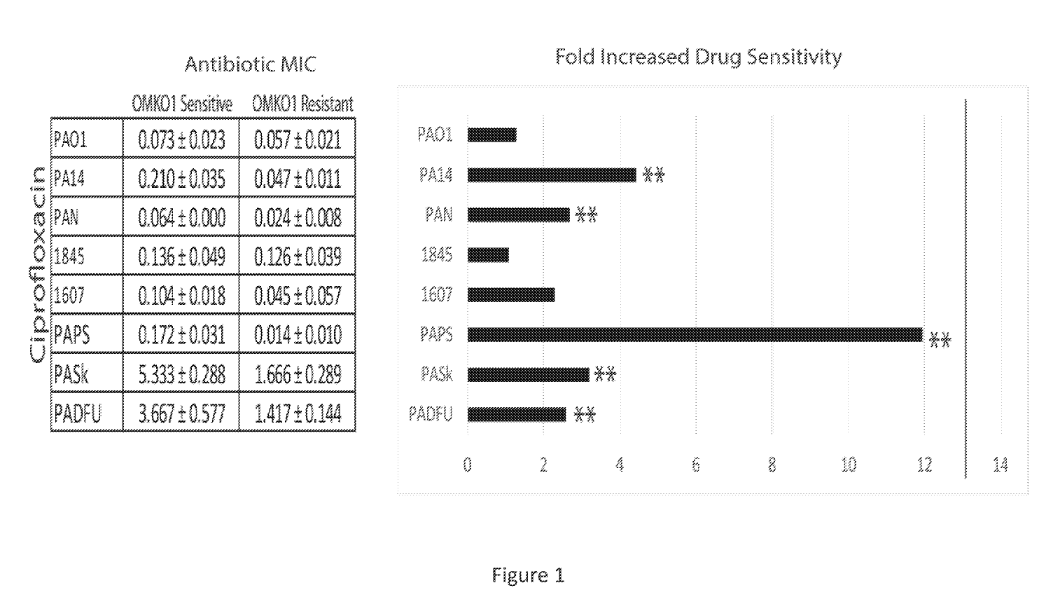

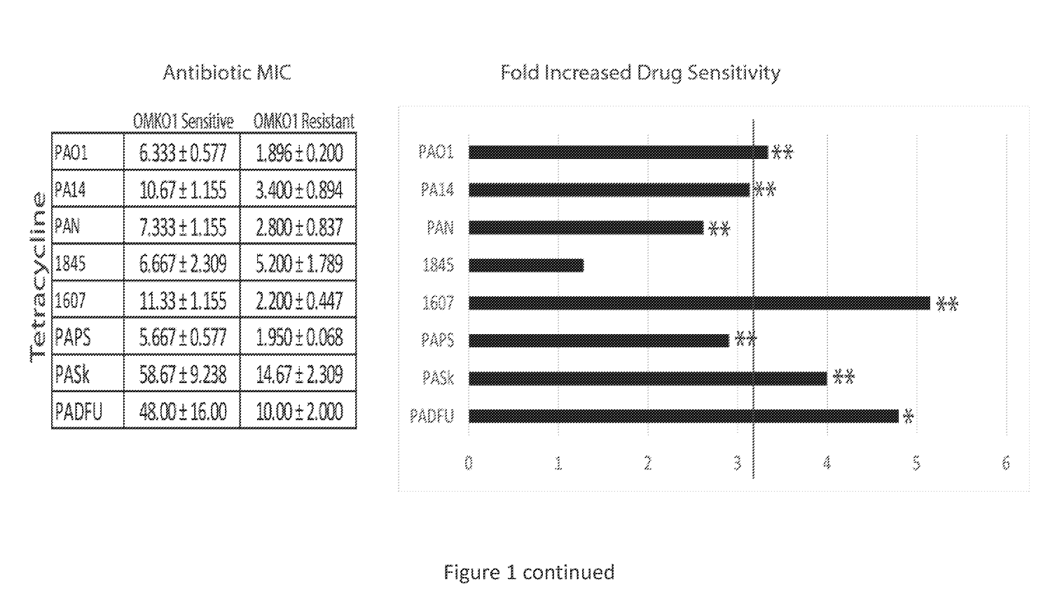

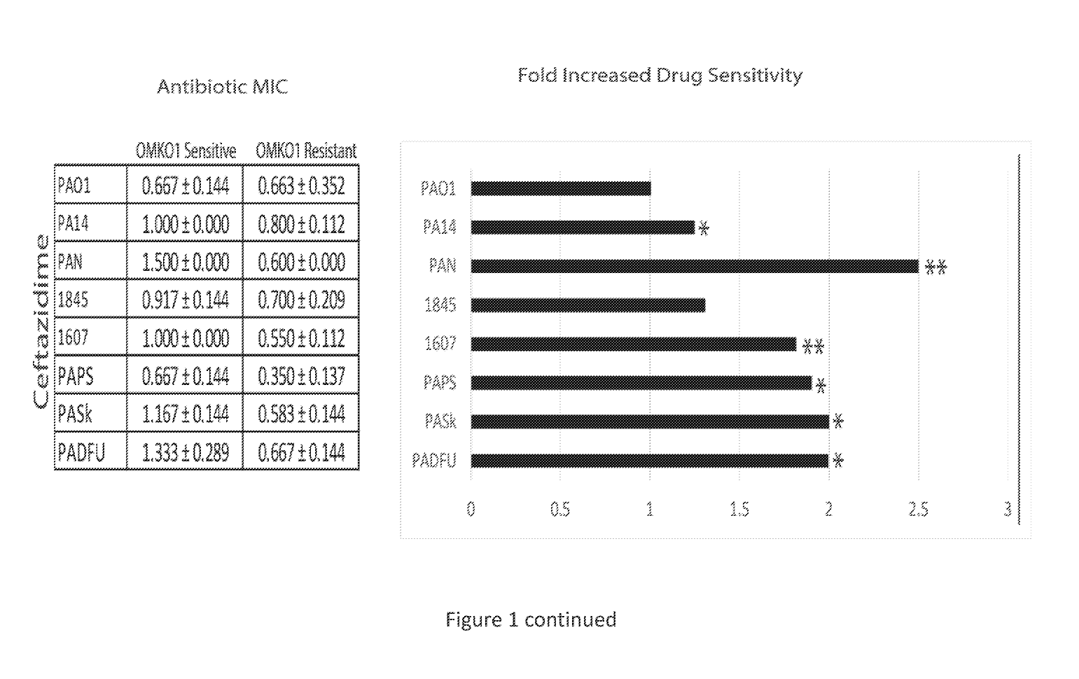

As one of the first classes of antimicrobials discovered in the modern era, the application of phages has had a controversial past and their clinical use has not been fully accepted in Westernized countries. However, studies performed in the latter half of the 20th century (Smith, H. W., et al., J Gen Microbiol. 129(8), 2659-75 (1983)) and recent clinical trials (Wright, A, et al., Clin Otolaryngol. 34(4), 349-57 (2009); Rhoads, D. D., et al., J Wound Care 18(6), 237-8, 240-3 (2009)) demonstrating safety and efficacy have renewed interest in phage therapy as a possible mechanism by which antibiotic resistant and biofilm-associated infections might be controlled. As a class of antibacterials, phages are distinct from traditional chemical antibiotics in four seemingly beneficial ways: they are self-amplifying/limiting in the presence/absence of substrate (i.e., susceptible bacteria); they are often able to penetrate biofilms to reach infectious bacteria; they are capable of infecting/killing persister cells; and their killing mechanism is distinct from those of traditional antibiotics. Exploiting the differences between antibiotics and phage therapy has been a driving force for continued research into the potential clinical utility of phage therapy. Phage OMKO1 has been identified that utilizes the outer membrane protein M of the mexAB- and mexXY-multidrug efflux systems of P. aeruginosa, forcing bacteria to trade acquisition of phage resistance for increased antibiotic sensitivity (FIG. 1). In other words, bacteria which develop resistance to phage OMKO1 by altering the binding sites of their efflux systems decrease their ability to extrude antibiotics and increase antibiotic sensitivity. Increased antibiotic resistance through the extrusion of antibiotics using these efflux systems conversely increases phage OMKO1 sensitivity. The use of this phage therapeutically has not been previously tested. An in vitro study examining the ability of phage OMKO1 to disrupt 72-hour biofilms grown on these materials was conducted to determine whether phage OMKO1 could be used therapeutically. Following these assays, in addition to assays demonstrating resensitization to antibiotics after evolved resistance to phage OMKO1, phage OMKO1 was prepared for therapeutic application. The results of these in vitro assays and subsequent application of phage OMKO1 therapeutically in a clinical case report of a patient with a chronic aortic graft infection are presented herein.

Also described herein is the use of bacteriophages to treat multi-drug resistant Pseudomonas aeruginosa infections on vascular grafts demonstrated through experimental studies on prosthetic material and a case report. Also disclosed herein is the use of bacteriophages to treat multi-drug resistant Pseudomonas aeruginosa infections in a murine model.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The Materials and Methods used in the performance of the experiments disclosed herein are now described.

Pseudomonas aeruginosa Strains:

P. aeruginosa strains PA01 and PA14 were kindly provided by B. Kazmierczak (Yale School of Medicine). Strains derived from PA01 that each contained a knockout of a gene in the Mex system were obtained from the Pseudomonas aeruginosa PA01 Transposon Mutant Library (Manoil Lab, University of Washington).

P. aeruginosa PAPS was collected from fistular discharge of a patient with a history of chronic infection associated with an aortic arch replacement surgery. This strain was associated with a biofilm that formed on an indwelling Dacron aortic arch and has been present for >1 year in the patient. P. aeruginosa PASk was collected from an open wound on the skull of a 60 y.o. male that was not responsive to antibiotic therapy or hyperbaric oxygen. P. aeruginosa PADFU was collected from a diabetic foot ulcer. These strains were collected from consented donors and de-identified. Furthermore, experiments were performed in accordance with The Yale University Human Investigation Committee/Institutional Review Board (HIC/IRB) guidelines, and relevant experimental protocols were approved by Yale's HIC/IRB committee.

P. aeruginosa strains 1845 and 1607 were collected from household sink drains (1845: bathroom sink drain; 1607: Kitchen sink drain; Remold, S. K. et al., Microbial Ecology 62, 505-517 (2011)), and kindly provided by S. Remold, University of Louisville.

Challenge Assays Using Knockout Library of P. aeruginosa:

The transposon knockout mutants used for screening included 11 strains, which differed in the knockout of a gene for a surface expressed protein in the Mex system: oprC, oprB, oprG, oprD, oprI, oprH, oprP, oprO, oprM, oprD, oprN. Also, phage ability to grow on 8 strains that differed in the knockout of a gene for an internal protein of the Mex system: mexH, mexA, mexB, mexR, mexC, mexD, mexE, mexF was tested. These replicated (n=3) assays calculated the average efficiency of plating (EOP) on a knockout host: plating ability (titer in plaque-forming units per mL) for a phage on the test knockout strain, relative to its plating ability on a phage sensitive host (PA01).

Isolation of Phage OMKO1:

The phage isolated from Dodge Pond was serially passaged on host strain PA01 for 20 consecutive passages. PA01 was grown to exponential phase in 25 ml of Luria-Bertani (LB) broth and then infected with phage at multiplicity of infection (MOI; ratio of phage particles to bacterial cells) of ˜0.1, using 37° C. shaking (100 rpm) incubation. After 12 hours, the culture was centrifuged and filtered (pore size: 0.22 μm) to remove bacteria, and to obtain a cell-free lysate. The next passage was initiated under identical conditions, using naïve (non-coevolving) PA01 bacteria grown fresh from frozen stock. This process was continued for 20 passages total, and phage OMKO1 was plaque purified from the endpoint phage population.

Isolation of Phage Resistant Mutants:

Phage OMKO1 was amplified on P. aeruginosa in liquid culture in conditions identical to the Serial passage assays. Following 12 hours of amplification, 100 μl of culture was plated on LB agar and incubated for 12 hours. Individual colony-forming units (CFUs) were then collected, and verified to be phage resistant by classic ‘spot tests’ (i.e., 10⁷ PFU of phage OMKO1 was pipetted onto a lawn of each bacterial isolate to test whether the phage was capable of visibly clearing the lawn [indicating bacterial sensitivity to phage] versus incapable of clearing the lawn [indicating bacterial resistance to phage]).

Minimum Inhibitory Concentration Assays:

Bacterial strains were grown overnight at 37° C. as described above. A 200 μL sample of the culture was then spread onto an LB agar plate, and allowed to dry for 10 minutes, followed by application of an eTestStrip (BioMerieux) for a test antibiotic. Plates were incubated at 37° C. for 12 hours, and MIC was estimated as the point at which bacterial growth intersected the eTest strip. Each strain was tested in triplicate for each antibiotic.

Bacterial Growth Kinetics:

Bacterial growth was assayed using a TECAN Freedom EVO workstation (TECAN Schweiz AG, Männedorf, Switzerland), which included an automated spectrophotometer (TECAN INFINITE F200 plate-reader) to monitor changes in bacterial density (optical density=OD₆₀₀) and a Robotic Manipulator Arm (RoMA) to manipulate cultures grown in 96-well flat-bottomed optical plates (Falcon). Each test strain was grown in LB broth with replication (n=3), and some assays included bacteria mixed with phage OMKO1 at an MOI˜10 to increase the probability that all susceptible bacteria in the well were initially infected. Assays were controlled via scripts prepared in TECAN's Freedom EVOWare and iControl software. Plate incubation occurred at 37° C. with 5 Hz continuous shaking in incubation ‘towers’. Every 2 min, each plate was sequentially transferred by the RoMA to the plate reader to measure OD. Within the plate reader, prior to OD reading the plate was shaken orbitally at 280 rpm and with 2 mm amplitude for 10 seconds. Absorbance wavelength was measured at 620 nm over the course of 15 flashes, and the resulting OD for each well was outputted by iControl into a time-stamped delimited text file, which was then imported to Excel (Microsoft) for further analysis. The plate was then transferred by RoMA back to the incubation tower, and the protocol was repeated for 12 hours total.

Bioinformatics Analysis:

Syntenic copies of the genes oprM, mexA, mexB, mexX and mexY were extracted from 38 publicly available genomes (Table 1) of P. aeruginosa, representing a cross section of the extant genetic diversity of the species. These sequences were aligned using MUSCLEv3.8.31⁴² and refined by eye. Maximum likelihood trees were estimated for each gene using RaxMLv8.0.0⁴³. The d_{N}/d_{S} (ω) ratio for each gene was calculated using the codeML of PAMLv4.8⁴⁴ using model M2a with co both fixed and variable. Significance of positive selection for each gene was evaluated by conducting a likelihood ratio test of the likelihood values implemented in the base package of R software v. 3.2.1.

TABLE 1
Summary of strains used for the selection analysis, including GenBank Assembly
number, source and country, date and isolation notes where known.

| | GenBank | | | Collection | |
|---|---|---|---|---|---|
| Strain Name | Accession | Source | Country | Date | Notes |
| VRFPA01 | GCA_000335395.3 | Sankara Nethralaya | India | 2012 | blood isolate from |
| | | Vision Research | | | Indian septicemia |
| | | Foundation | | | patient |
| AZPAE15042 | GCA_000790465.1 | AstraZeneca | USA | unknown | cystic fibrosis isolate |
| PA7 | GCA_000017205.1 | J. Craig Venter | Argentina | unknown | non-respiratory |
| | | Institute | | | clinical isolate |
| 19660 | GCA_000481765.1 | Broad Institute | USA | unknown | Cornea/ocular |
| | | | | | infection |
| 19br | GCA_000223945.2 | IBIS, Universite | unknown | unknown | unknown |
| | | Laval | | | |
| AZPAE14903 | GCA_000791145.1 | AstraZeneca | Spain | 2008 | itra-abdominal tract |
| | | | | | infection |
| P2-L230/95 | GCA_000760505.2 | Center for Cellular | India | 1995 | Obtained from |
| | | and Molecular | | | Keratitis Patient |
| | | Biology(CCMB) | | | |
| PA38182 | GCA_000531435.1 | University of London | UK | unknown | resistant to |
| | | | | | antibiotics other than |
| | | | | | colistin |
| X24509 | GCA_000481865.1 | Broad Institute | USA | unknown | UTI patient |
| BWHPSA028 | GCA_000481145.1 | Broad Institute | USA | 2013 | Isolated from |
| | | | | | Sputum |
| C41 | GCA_000480455.1 | Broad Institute | unknown | unknown | Environmental |
| | | | | | isolate |
| CF27 | GCA_000481905.1 | Broad Institute | USA | unknown | Cystic fibrosis |
| | | | | | patient |
| BWHPSA022 | GCA_000481265.1 | Broad Institute | USA | 2013 | Sputum isolated |
| DQ8 | GCA_000283055.1 | Shanghai Jiao Tong | China | unknown | soil isolated |
| | | University | | | |
| PA01 | GCA_000006765.1 | PathoGenesis | Australia | 1955 | wound isolated |
| | | Corporation | | | |
| PDR | GCA_000783275.1 | China CDC | China | unknown | isolate from a patient |
| | | | | | with urinary infection |
| BWHPSA037 | GCA_000520455.1 | Broad Institute | USA | 2013 | bronchoalveolar |
| | | | | | lavage |
| CF77 | GCA_000480375.1 | Broad Institute | USA | 2005 | cystic fibrosis isolate |
| LESB58 | GCA_000026645.1 | Wellcome Trust | UK | 1988 | cystic fibrosis isolate |
| | | Sanger Institute | | | |
| NCMG1179 | GCA_000291745.1 | National Center for | Japan | 2010 | isolated from |
| | | Global Health and | | | inpatient respiratory |
| | | Medicine | | | tract |
| AZPAE14698 | GCA_000794705.1 | AstraZeneca | Israel | unknown | respiratory tract |
| | | | | | infection |
| C23 | GCA_000480495.1 | Broad Institute | unknown | unknown | Environmental |
| | | | | | isolate |
| PS42 | GCA_000520195.1 | Broad Institute | Venezuela | unknown | Environmental |
| | | | | | isolate |
| Stone130 | GCA_000478465.2 | Broad Institute | unknown | unknown | unknown |
| B13633 | GCA_000359505.1 | National Tsing Hua | unknown | unknown | infant with |
| | | University | | | community-acquired |
| | | | | | diarrhea |
| VRFPA04 | GCA_000473745.3 | Vision Research | India | unknown | Isolated from Human |
| | | Foundation, Sankara | | | corneal button |
| | | Nethralaya | | | |
| BL04 | GCA_000481065.1 | Broad Institute | USA | unknown | isolated from eye |
| PABL056 | GCA_000290555.1 | Northwestern | USA | 2001 | isolated from blood |
| | | University Feinberg | | | |
| | | School of Medicine | | | |
| BL13 | GCA_000480885.1 | Broad Institute | USA | unknown | isolated from eye |
| P7L63396 | GCA_000760495.2 | Center for Cellular | India | 1996 | isolate from keratitis |
| | | and Molecular | | | patient |
| | | Biology(CCMB) | | | |
| VRFPA03 | GCA_000467675.1 | Vision Research | India | 2012 | Corneal button from |
| | | Foundation, Sankara | | | corneal keratitis |
| | | Nethralaya | | | patient |
| VRFPA09 | GCA_000558345.1 | Vision Research | India | 2013 | blood sample from |
| | | Foundation, Sankara | | | patient with |
| | | Nethralaya | | | septicaemia |
| BL03 | GCA_000481085.1 | Broad Institute | USA | unknown | Corneal Scaping |
| 39016 | GCA_000148745.1 | Centre for Genomics | unknown | unknown | cornea of a patient |
| | | Research, University | | | with ulcerative |
| | | of Liverpool | | | keratitis |
| AZPAE13850 | GCA_000795435.1 | AstraZeneca | India | unknown | unknown |
| BL25 | GCA_000480645.1 | Broad Institute | USA | unknown | isolated from eye |
| AZPAE14699 | GCA_000794725.1 | AstraZeneca | USA | 2012 | itra-abdominal tract |
| | | | | | infection |
| UCBPPPA14 | GCA_000014625.1 | Massachusetts | USA | unknown | Human clinical |
| | | General Hospital | | | isolate |

Biofilm Elimination Assays:

Laboratory assays were performed to examine the impact of phage OMKO1, on 72-hour-old P. aeruginosa biofilms grown on Dacron and/or other prosthetic material(s). Biofilms were grown on 3 mm×3 mm sections of Dacron, Gore-Tex, felt or 3 mm lengths of polypropylene sutures, by inoculating each material in 150 μL 0.1×LB broth in a 96-well dish with 50 μL of an overnight culture of P. aeruginosa isolated from fistular discharge of a case report patient. Overnight cultures of this strain had a cell density of 10⁹ colony forming units (CFU) per ml, consistent with other laboratory strains of P. aeruginosa. Test pieces were removed from this dilute growth media after 72-hours and rinsed with 200 μL of 0.1×LB three times to remove planktonic cells. Dilute growth medium was utilized in order to induce biofilm formation. Following rinse, sections were added to 200 μL of LB medium containing treatment (phage OMKO1, ceftazidime or ciprofloxacin at 2×MIC, antibiotic at 2×MIC+phage OMKO1, or blank control). Following exposure to treatment for 24 hours, sections were placed in fresh LB medium and allowed to incubate at 37° C. for an additional 24 hours without agitation. Sections were then removed and cell density was measured with an automated spectrophotometer (Tecan model Infinite F200 microplate-reader). Explicit care was taken during each rinse and transfer to ensure sterile conditions and prevention of cross-contamination between replicates and treatment groups.

Minimum Bactericidal Titer:

The minimum bactericidal titer of phage OMKO1 was determined using methods identical to the biofilm eradication assays conducted in 96-well dishes and was applied to 3 mm×3 mm sections of Dacron. Treating bacteria with phage in this assay comprised serial 10-fold dilutions of phage OMKO1 starting at 10¹⁰ plaque forming units (PFU) per mL. Each treatment consisted of adding 10 μl of phage OMKO1 from the appropriate dilution to a well containing 72-hour biofilms grown in identical conditions to the biofilms elimination assays. Phage density ranged from 10⁸ PFU/well down to approximately 10 PFU/well. The assay was performed similar to the biofilm eradication assay. After treatment, cell growth was measured with an automated spectrophotometer allowing for determination of the minimum multiplicity of infection (MOI: phage OMKO1 particles per bacterium) required to eradicate biofilms on Dacron sections.

Purification and Preparation of Phage OMKO1:

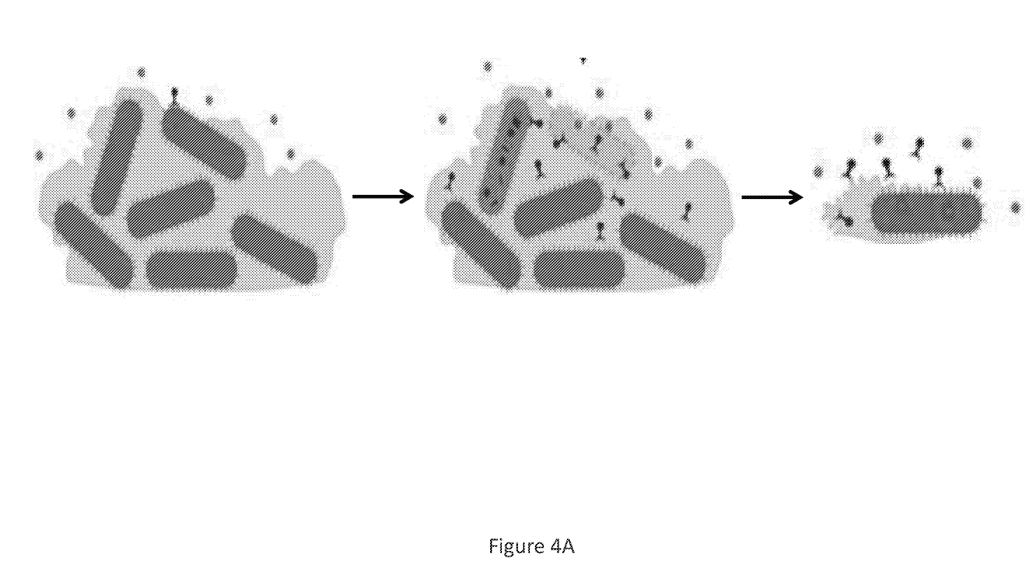

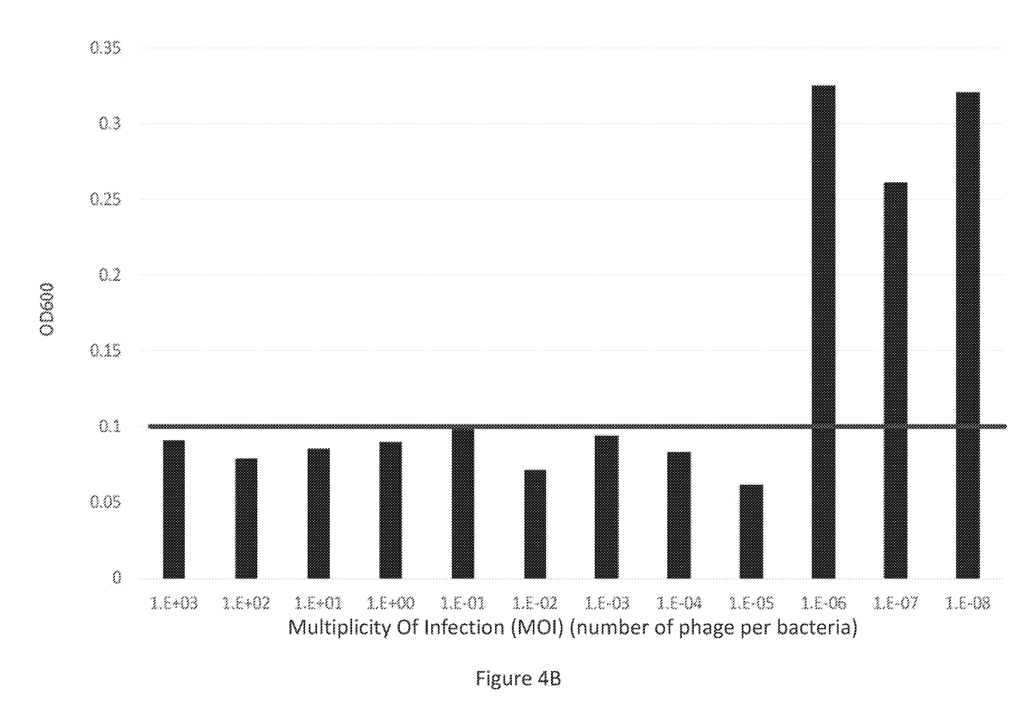

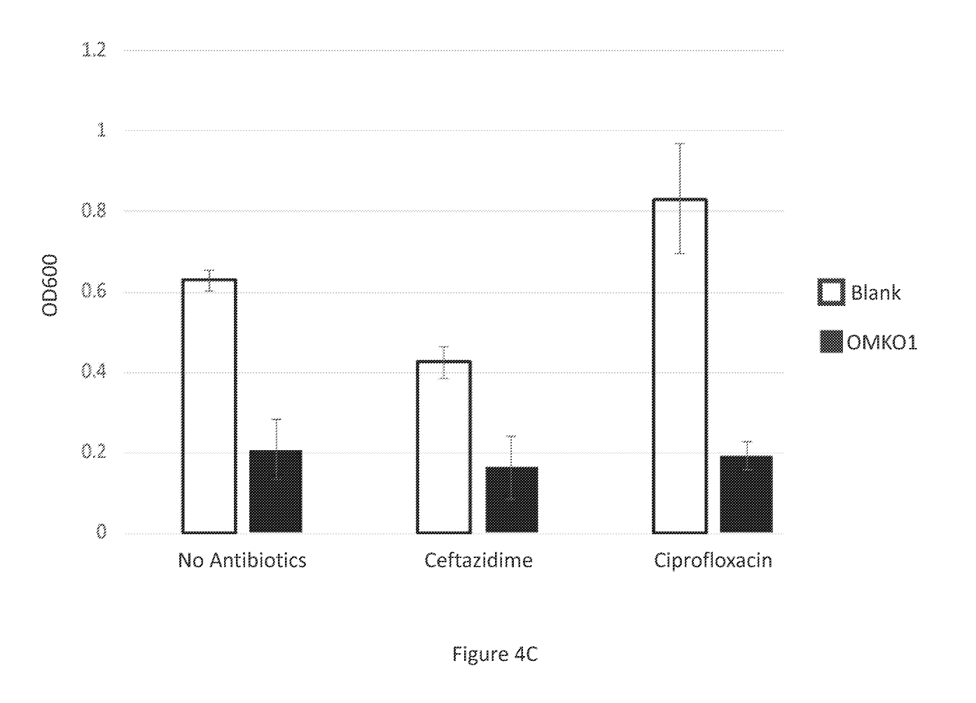

Use of phage OMKO1 in any assay required removal of endotoxins present in phage lysate. This was accomplished via spin column (Pierce High Capacity Endotoxin Removal Spin Columns, ThermoFischer) followed by dialysis in phosphate buffered saline. Limulus amebocyte lysate (LAL) testing was then performed by a third party laboratory (Associates of Cape Cod, East Falmouth, Mass.) to determine endotoxin concentrations. Upon receiving endotoxin levels, dilution of the preparation was then performed in injectable saline to produce a concentration of 12.5 EU/mL and final titer of 10⁷ plaque forming units per mL (PFU/mL). This titer was determined to be acceptable, and much greater than the minimum bactericidal titer determined previously for eradication of 72 hour biofilms (FIG. 4).

Strain Characterization:

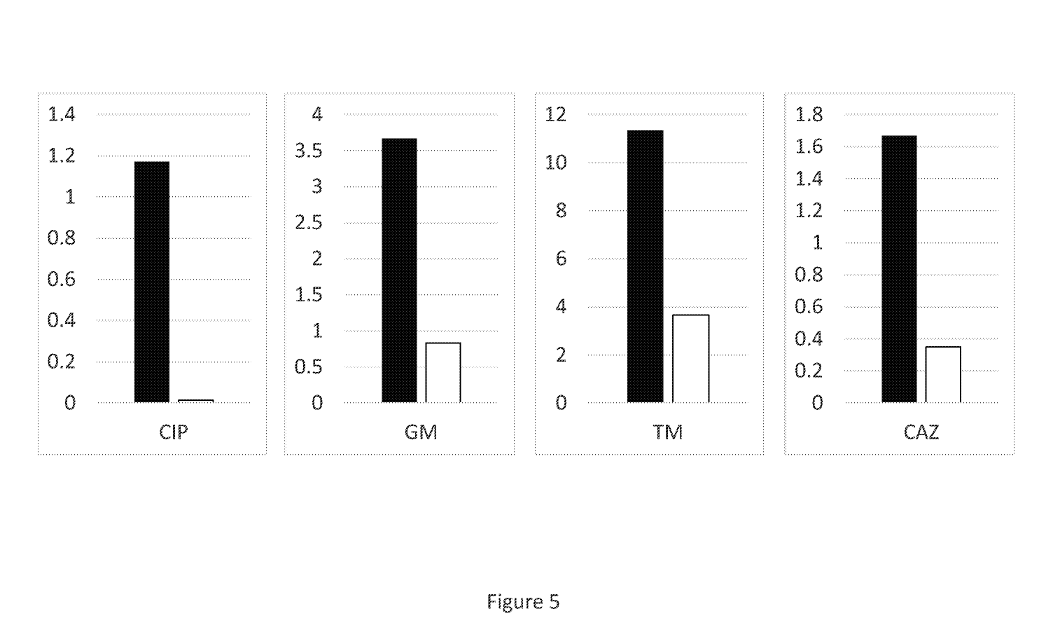

P. aeruginosa isolated from the patient was subjected to Deep Sequencing (Next Generation Sequencing) on the Illumina HiSeq 2000 platform to confirm species identity and presence of genes known to be associated with efflux-pump mechanisms for resistance to antibiotics. Furthermore, a trade-off between phage OMKO1 sensitivity and antibiotic susceptibility was confirmed as per methods disclosed elsewhere herein (FIG. 5). Phage OMKO1 was sequenced also on the HiSeq 2000 platform and found to be in the PhiKZ-like virus genus.

Case Report Example:

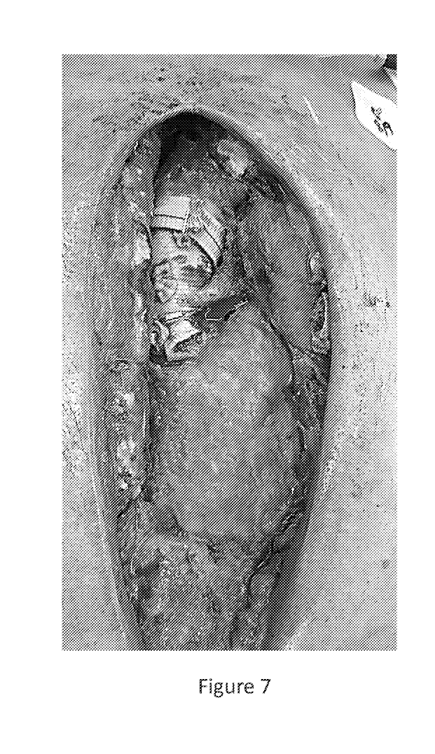

In July 2012, a 76 year old male underwent a coronary artery bypass and aortic arch replacement surgery. This was complicated by a subsequent mediastinal and graft infection (FIG. 7). The patient returned to the operating room several times for debridement and washout of the infected chest wall and eventually closure of the mediastinum with an omental and bilateral pectoralis major flaps. Eventually, the aortic graft became chronically infected, and the patient went on to develop a thoracic abscess and associated fistula to the chest wall. The fistula spontaneously expressed purulent fluid, and P. aeruginosa grew on repeated cultures from this expressed fluid. The patient was placed on oral ciprofloxacin based on susceptibility testing but had several episodes of bacteremia for which he was admitted and treated with intravenous ceftazidime. He went on to receive solely intravenous ceftazidime for nearly two years which suppressed the patient's aortic graft infection but was unable to completely clear it. Because of the patient's surgical history and current medical condition, further elective surgical management was not an option due to the high mortality risk. The patient wished to explore other options aside from indefinite antibacterial treatment and it was deemed at this time that the patient would make an ideal candidate for exploration of phage therapy. A procedure was proposed in which the patient's thoracic abscess would be directly accessed by needle puncture using image guidance to distribute a mixture of phage OMKO1 and ceftazidime. After the risks and benefits of the experimental procedure were discussed, the patient consented to the procedure. The Food and Drug Administration and Yale University Human Investigation Committee gave their approval for the use of phage OMKO1 as an investigational new drug (FDA IND#16827).

A sampling of the fistular discharge was obtained. The thoracic abscess was accessed through direct needle puncture using image guidance. The needle was withdrawn from the chest and 10 mL of phage OMKO1 (10⁷ PFU/mL) and ceftazidime (0.2 g/mL) solution was topically applied into the anterior chest fistula. A sterile dressing was placed over the fistula and the patient was admitted to a telemetry monitored bed from where he was discharged with stable vital signs. Approximately five weeks after the procedure the patient underwent emergency partial removal of the Dacron graft. Cultures were taken at the time of the operative intervention.

Phage Recovered from Experimental Mice in NIH Preclinical Services Study:

A small-scale efficacy trial in a murine model of acute lung pneumonia was performed in collaboration with NIH/NIAID contracted researchers at University of Louisville. The murine model (Lawrenz et al., FEMS Pathogens and Disease, 2015; 73) was used to test whether phage OMKO1 is effective in combating lung infection by Pseudomonas aeruginosa strain UNC-D. Treatments contained bacteria-infected mice that were also given a dose of the phage alone, phage plus antibiotic, or antibiotic alone. Tissue samples (lung, spleen) were collected from each of the experimentally-infected mice in the study. In this short duration model, mice were sacrificed roughly 30 hours post infection. The tissue samples were subjected to classic microbiology assays, to attempt isolation of phage particles; this effort was successful in samples from mice that received phage therapy.

The Results of the experiments disclosed herein are now described.

Increasing prevalence and severity of multi-drug-resistant (MDR) bacterial infections has necessitated novel antibacterial strategies. Ideally, new approaches would target bacterial pathogens while exerting selection for reduced pathogenesis when these bacteria inevitably evolve resistance to therapeutic intervention. As an example of such a management strategy, a lytic bacteriophage, OMKO1, (family Myoviridae) of Pseudomonas aeruginosa that utilizes the outer membrane porin M (OprM) of the multidrug efflux systems MexAB and MexXY as a receptor-binding site was isolated. Results showed that phage selection produced an evolutionary trade-off in MDR P. aeruginosa, whereby the evolution of bacterial resistance to phage attack changed the efflux pump mechanism causing increased sensitivity to drugs from several antibiotic classes. Although modern phage therapy is still in its infancy, it is concluded that phages, such as OMKO1, represent a new approach to phage therapy where bacteriophages exert selection of MDR bacteria to become increasingly sensitive to traditional antibiotics. This approach, using phages as targeted antibacterials, could extend the lifetime of the current antibiotics and potentially reduce the incidence of antibiotic resistant infections.

Described herein is an evolutionary-based strategy that forces a genetic trade-off by utilizing phages that drive MDR bacterial pathogens to evolve increased phage resistance by suffering increased sensitivity to chemical antibiotics. Thus, this approach to phage therapy should be doubly effective; success is achieved when phage lyse the target bacterium, and success is also achieved when bacteria evolve phage resistance because they suffer increased sensitivity to antibiotics. It is shown herein that phage capable of binding to surface-exposed OprM of the MexAB and MexXY systems of MDR P. aeruginosa exert selection for bacteria to evolve phage resistance, while impairing the relative effectiveness of these efflux pumps to extrude chemical antibiotics.

Samples were obtained from six natural sources (sewage, soil, lakes, rivers, streams, compost) and enriched for phages that could infect P. aeruginosa strains PA01 and PA14, two widely used MDR P. aeruginosa models. This effort yielded 42 naturally isolated phages that successfully infected both strains of MDR P. aeruginosa.

To test if any of these phages could bind to OprM of MexAB and MexXY efflux systems, a transposon knockout collection of bacterial mutants derived from P. aeruginosa strain PA01 was used. The assays described herein determined which bacterial mutants failed to support phage infection, because such mutants lacked the surface-expressed protein necessary for phage infection. The assays measured the efficiency of plating (EOP), defined as the ratio of phage titer (plaque-forming units [pfu] per mL) on the knockout host relative to titer on the unaltered PA01 host. EOP≈1.0 indicated that the protein associated with the knocked out gene was irrelevant for phage binding, whereas EOP=0 implicated the knocked out protein as necessary for infection.

Results showed that one of the 42 phage isolates failed to infect the ΔoprM knockout strain, but successfully infected wildtype PA01 and all other tested knockout mutants. This phage was originally isolated from a freshwater lake sample (Dodge Pond, East Lyme, Conn., USA).

The phage was then experimentally evolved on P. aeruginosa strain PA01 for 20 consecutive passages, where each passage consisted of 24-hour growth on naïve (non co-evolved) bacteria grown overnight from frozen stock. This design selected for generalized improvement in phage growth but prevented the possibility for host co-evolution. Following serial passage, a plaque-purified sample was isolated from the evolved phage population to obtain strain OMKO1 (i.e., outer-membrane-porin M knockout dependent phage #1). A whole-genome sequencing analysis was conducted of this clone and determined that phage OMKO1 had a genome size of ˜278 kb (GenBank accession number pending) and belonged to the dsDNA virus family Myoviridae (genus: phiKZ-like-viruses).

It was next tested whether resistance to phage OMKO1 caused the desired genetic trade-off between phage resistance and antibiotic sensitivity in MDR P. aeruginosa. In particular, it was determined whether phage resistance allowed improved killing efficiency (decreased minimum inhibitory concentration; MIC) of four antibiotics, representing four drug classes of varying capacity for efflux via MexAB and/or MexXY-OprM: Ceftazidime (CAZ), Ciprofloxacin (CIP), Tetracycline (TET), and Erythromycin (EM). CAZ is effluxed by the Mex system, but resistance is also inducible, determined by genetically encoded β-lactamases. CIP resistance can also be regulated by multiple factors such as mutations in DNA gyrase or topoisomerase IV in addition to efflux. However, resistance to TET and EM is primarily due to efflux via the MexAB- and MexXY-OprM efflux systems.

The effects of phage resistance on sensitivity to the four drugs was tested in replicated assays with PA01 and PA14, as well as with three environmental strains (PAN, 1607, 1845) and three clinical isolates (PAPS, PASk, PADFU). In these assays, the phage-OMKO1 resistant strain was either a knockout mutant (ΔoprM derived from PA01), or an independently derived spontaneous mutant of the associated parental strain.

Results for strain PA01 are shown in FIG. 1. In comparison, strain PA01 ΔoprM showed increased average drug sensitivity relative to PA01, in the two antibiotic environments where Mex systems provide primary (TET: 2.00±0.00 μg/mL; EM: 4.667±0.00 μg/mL) or moderate (CIP: 0.016±0.00 μg/mL; CAZ: 0.210±0.144 μg/mL) drug resistance. Thus, loss of OprM expression provided resistance to phage OMKO1, but caused greater sensitivity to all four drugs (Fold Increased Sensitivity to TET, CAZ, and EM: p<0.01; CIP: p<0.05) (cf. FIG. 1). The ratio of mean MIC for PA01 relative to that for ΔoprM was used to estimate the fold increased drug sensitivity associated with phage resistance (FIG. 1), which may be considered a baseline improvement in drug efficacy upon acquisition of phage resistance. Similar results were observed for a spontaneous phage-OMKO1 mutant of PA01 when Mex systems provided primary resistance (TET and EM; FIG. 1).

As a control for transposon insertion, strain ΔmexR, which was also derived from PA01, was examined. mexR, the repressor of MexAB-OprM and MexXY-OprM operons should not negatively alter phage sensitivity. As expected, this control strain was phage sensitive and the MIC assays showed inhibitory antibiotic concentrations equivalent or higher than PA01 (TET: 256.00±0.00 μg/mL; EM: 256.00±0.577 μg/mL; CIP: 32.00±0.00 μg/mL; CAZ: 1.333±0.035 μg/mL), confirming that over-expression of Mex systems improved growth in antibiotic environments where PA01 showed drug sensitivity.

In addition, the trade-off hypothesis was examined in model strain PA14, for all four drugs. Spontaneous phage resistance caused a statistically significant fold-increase in antibiotic sensitivity (FIG. 1). Altogether, these data showed that phage resistance led to greater drug sensitivity for antibiotics primarily controlled by Mex systems, but only sometimes improved drug efficacy when Mex systems exerted less control.

Model strains PA01 and PA14, and knockout mutants derived from these strains, are useful for elucidating mechanisms such as phage binding targets. However, microbial models inevitably experience some selection for improved fitness under controlled lab conditions, creating a potential divergence from more recently isolated clinical and environmental samples. Thus, experiments were designed to confirm whether the desired trade-off between phage-OMKO1 resistance and increased drug sensitivity occurred in environmental and clinical strains.

After determining that the clinical and environmental strains were sensitive to phage OMKO1, spontaneous phage-resistant mutants of each strain were isolated, and MIC assays were conducted. Results (FIG. 1) confirmed that resistance to phage OMKO1 coincided with increased sensitivity of each environmental isolate to antibiotics TET and EM. Phage resistance led to greater drug sensitivity for two clinically relevant antibiotics (CAZ, CIP), with the majority of outcomes showing statistical significance (FIG. 1). Importantly, the phage resistant mutants of all three of the clinical isolates (PAPS, PASk, PADFU) showed significantly increased drug sensitivity to the tested antibiotics. Thus, results for the environmental and clinical isolates qualitatively matched those observed in the well-characterized strains PA01 and PA14, suggesting that phage OMK01 is capable of forcing the desired genetic trade-off in MDR P. aeruginosa.

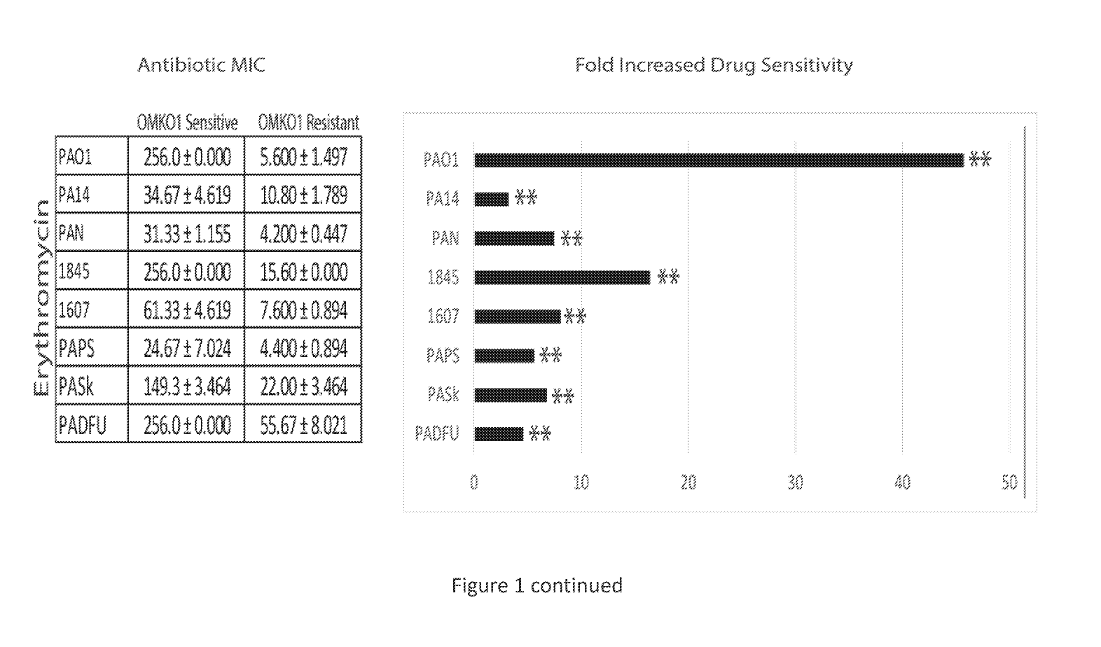

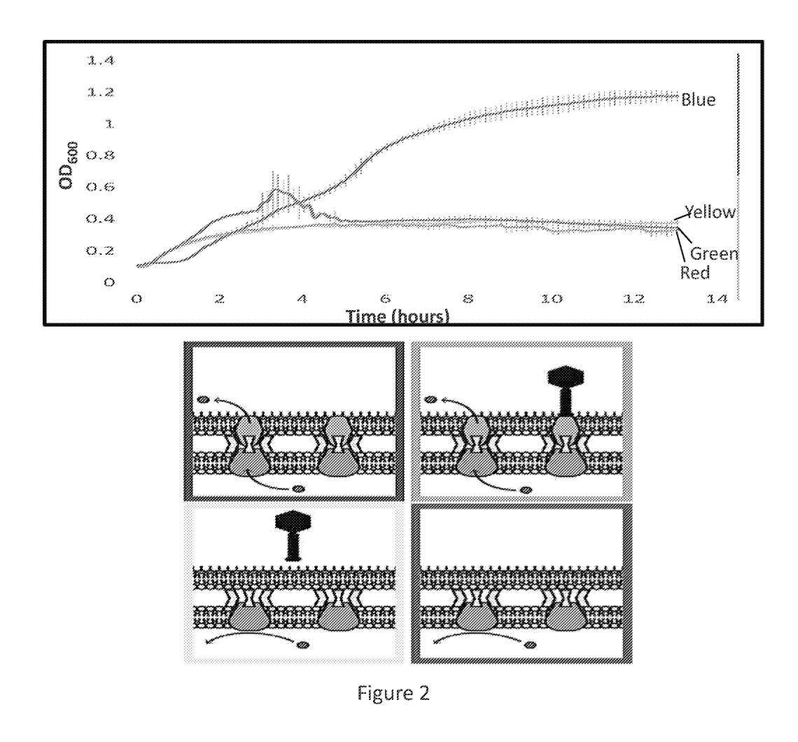

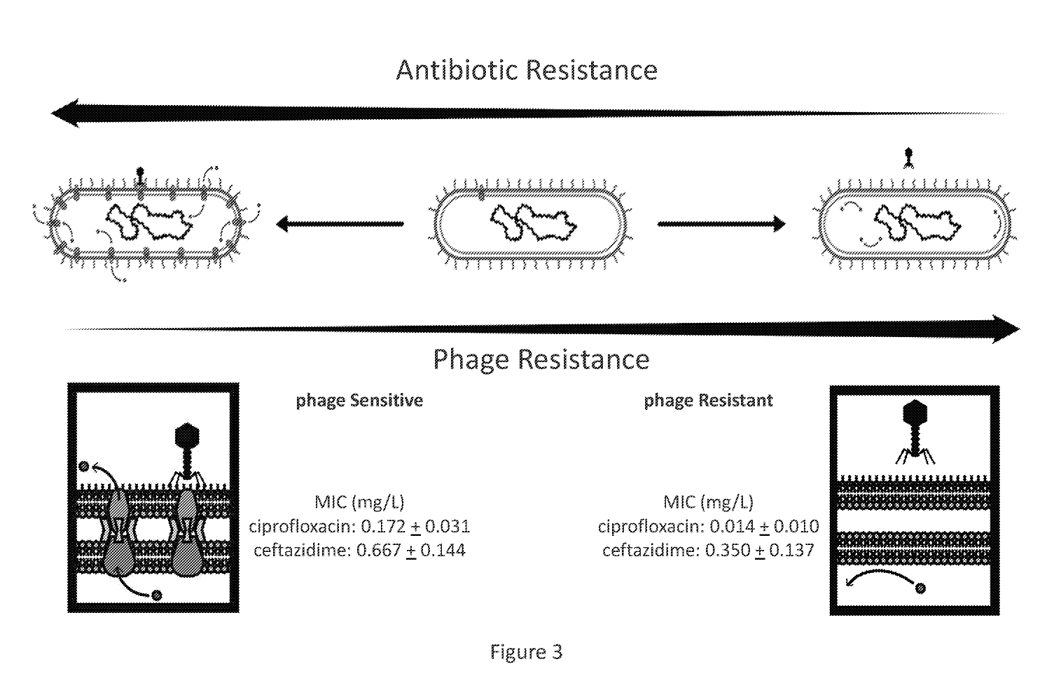

The effects of phage sensitivity versus resistance on P. aeruginosa fitness was further compared by examining growth kinetics of bacterial mutants in antibiotic medium when phage OMK01 was either present or absent. Bacterial growth curves were obtained by monitoring changes in optical density (OD₆₀₀) in liquid culture. These assays challenged knockout strains ΔmexR and ΔoprM to grow in a TET (10 μg/mL) environment, where phage OMK01 was either present or absent. Results (FIG. 2) confirmed that over-expression of Mex systems allowed robust growth of populations founded by strain ΔmexR in the presence of TET.

However, as expected these phage sensitive populations grew three-fold worse and highly similar to the ΔoprM population in an identical drug environment containing phage OMK01 (FIG. 2). Phage presence did not completely eliminate ΔmexR bacteria, perhaps explained by persistence of spontaneous phage resistant mutants that suffered the desired trade-off and failed to increase in density during the assay.

Phage resistant populations founded by strain ΔoprM showed impaired growth in the TET environment due to the knocked out OprM component of the Mex system. As expected, presence of phage OMK01 had no effect on growth kinetics of ΔoprM populations, because the virus was incapable of binding to these cells. In both cases, the observed weak growth of ΔoprM populations in TET environments was perhaps due to the low permeability ofP. aeruginosa cell membranes, which is problematic for treatment of these infections using antibiotics alone.

To evaluate whether phage OMKO1 would be broadly useful in targeting P. aeruginosa strains, the conservation of the MexAB- and MexXY-OprM efflux systems was examined. To do so, effects of selection on five genes encoded in these Mex systems (oprM, mexA, mexB, mexX, mexY) were estimated using genetic data from 38 P. aeruginosa strains representing the known genetic diversity of P. aeruginosa queried from NCBI GenBank. For each gene, this analysis measured ω (d_{N}/d_{S}): the ratio of the number of non-synonymous substitutions per non-synonymous site (d_{N}) to the number of synonymous substitutions per synonymous site (d_{S}), which is used to indicate selective pressure acting on a protein-coding gene.

Results (Table 2) showed that strong stabilizing selection was acting on oprM, mexA, mexB, and mexX genes, such that none of these loci were observed to be changing under positive selection. These data indicated that the structure of the OprM protein was strongly constrained to remain stable through time. Thus, phage OMK01 should be capable of infecting a wide variety of P. aeruginosa genotypes due to genetic stability of the binding target.

Furthermore, the analysis suggested low probability of wildtype functionality for novel mutations that would confer P. aeruginosa resistance to phage OMK01 via alteration of the OprM attachment site. A strong positive selection was detected only for P. aeruginosa gene mexY, for unknown reasons but indicating that this component of MexXY was changing relatively rapidly.

TABLE 2
Evaluation of selection acting upon genes associated with
MexXY- and MexAB-OprM efflux systems of P. aeruginosa.

| Gene | | p₀ | p₁ | p₂ = 1 − p₀ − p₁ | ΔLRT | p |
|---|---|---|---|---|---|---|
| oprM | p | 0.996 | 0.001 | 0.003 | 0.000 | 1.000 |
| | ω | 0.010 | 1.000 | 1.000 | | |
| mexA | p | 1.000 | 0.000 | 0.000 | 0.000 | 1.000 |
| | ω | 0.005 | 1.000 | 1.000 | | |
| mexB | p | 0.989 | 0.000 | 0.011 | 2.677 | 0.102 |
| | ω | 0.004 | 1.000 | 1.000 | | |
| mexX | p | 0.971 | 0.014 | 0.015 | 3.631 | 0.057 |
| | ω | 0.023 | 1.000 | 1.000 | | |
| mexY | p | 0.316 | 0.636 | 0.048 | 170.230 | <0.001 |
| | ω | 0.000 | 1.000 | 17.849 | | |

A bioinformatics approach tested whether data from published P. aeruginosa genomes showed conservation among oprM, mexAB and mexXY genes. p: proportion of variable sites assigned to each class in the selection model, ω: the overall d_{N}/d_{S} ratio for the variable sites which fit into each site class for each alignment. Selection models P₀: purifying selection, P₁: neutral selection: P₂: positive selection. ΔLRT: the difference in likelihood ratios between fixed and estimated ω values and associated p value for the presence of positive selection.

The results described herein show that phage OMKO1 is a naturally occurring virus that forces a desired genetic trade-off between phage resistance and antibiotic sensitivity. This trade-off benefits phage therapy efforts against MDR bacteria such as P. aeruginosa. Isolation of phage OMKO1 from nature suggested that other phages might have evolved to utilize OprM or other surface-exposed proteins of Mex systems as binding sites. These types of phage could be highly useful for developing therapeutics, because target bacteria are expected to inevitably evolve phage resistance resulting in antibiotic susceptibility.

Phage OMKO1 is the first evolutionary-based phage adjunctive, and this system exploits a genetic trade-off between phage and antibiotic resistance. The clinical utility of phages, such as OMKO1, is vital because selection using this phage restores usefulness of antibiotics that are no longer considered to be therapeutically valuable.

In the past, there was an attempt to restore waning amoxicillin efficacy, by combining this drug with clavulanic acid (a (3-lactamase inhibitor). Although clavulanic acid has minimal antibacterial activity, it interacts with β-lactamase enzyme via mechanism-based inhibition, allowing amoxicillin to inhibit cell wall synthesis. While this therapeutic approach often can be effective as demonstrated by more than 30 years of successful use of amoxicillin/clavulanic acid, the negligible antibacterial activity of clavulanic acid exerts selection pressure for hyper-production of β-lactamase as a means for bacteria to successfully evolve resistance to the adverse effects of clavulanic acid.

In contrast, the phage therapy approach described herein exerts selection pressure in the desired direction, causing bacteria to become increasingly antibiotic sensitive and allowing for renewed use of historically effective antibiotics that have been rendered useless by the evolution of antibiotic resistance. Furthermore, this approach suggests that antibiotics not typically used during treatment of P. aeruginosa infections due to intrinsic resistance could be used with phage OMKO1. This method effectively ‘re-discovers’ a class of antibiotics that has already been clinically tested/approved. Consequently, this approach has the potential to extend the effective lifetime of available antibiotics and broaden the spectrum of these drugs, greatly reducing the burden on drugs of last resort, preserving them for future use. Ideally, phage therapy that utilizes phages, such as OMK01, would not only improve clinical efficacy against MDR bacteria, but also potentially slow or reverse the incidence of antibiotic resistant bacterial pathogens.

Biofilm Elimination Assays

Briefly, OMKO1 disrupted P. aeruginosa biofilms and improved P. aeruginosa susceptibility to antibiotics. Additionally, OMK01 was applied clinically to treat a patient with a chronic P. aeruginosa infection associated with an aortic Dacron graft. After a single application of phages and ceftazidime, the patient has been off antibiotics for at least the past nine months with no signs of recurrent infection.

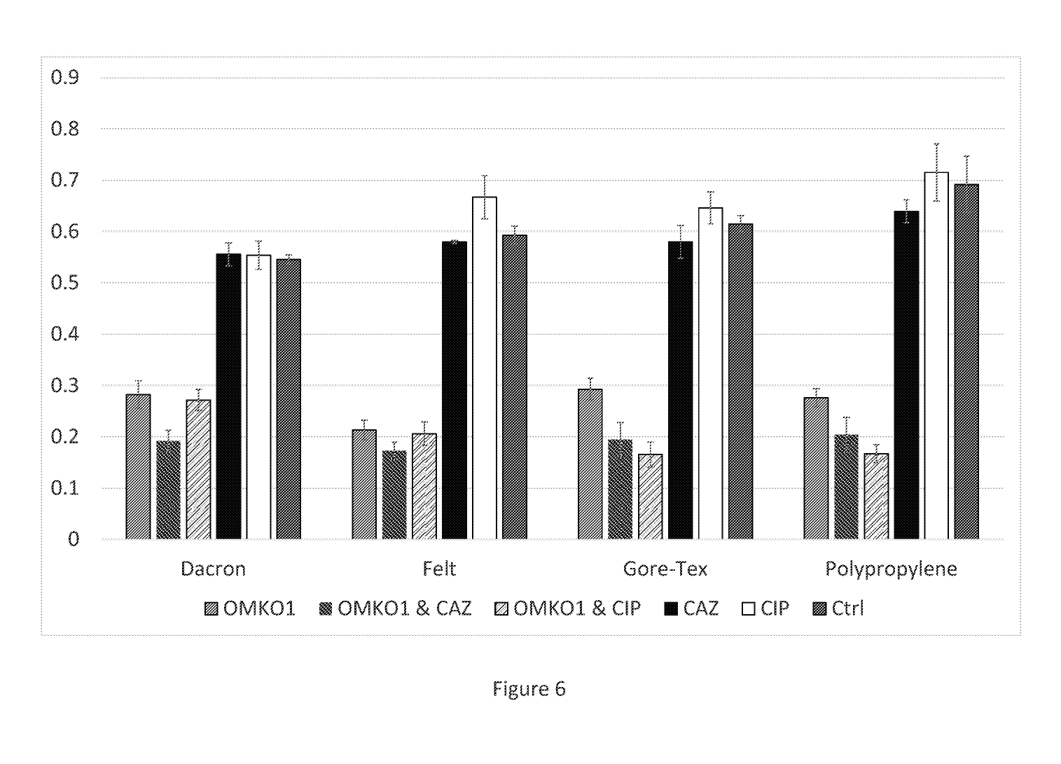

In detail, addition of phage OMK01 to either ciprofloxacin or ceftazidime eliminated biofilms grown on Dacron sections (p=0.0007, ciprofloxacin and p=0.001, ceftazidime) and was, itself significantly different than no treatment (p=0.003) (FIG. 6). Neither ciprofloxacin nor ceftazidime at 2×MIC was sufficient to eliminate 72 hour biofilms (p=0.73, ciprofloxacin; p=0.52, ceftazidime). Felt pieces had similar eradication effects and OMKO1 alone (p=0.003) or addition of OMKO1 to ciprofloxacin (p=0.003) or ceftazidime (p=0.001) were significant and neither antibiotic alone significantly removed 72-hour biofilms (p=0.147 ciprofloxacin, p=0.325 ceftazidime). Phage OMKO1 also significantly removed biofilms from Gore-Tex sections when used alone (p=0.003) or in combination with ciprofloxacin (p=0.0001) or ceftazidime (0.004), however, antibiotics alone were again unable to remove 72-hour biofilms at 2×MIC (p=0.289, ciprofloxacin, p=0.189, ceftazidime). Sections of polypropylene suture were also comparably cleared by phage OMKO1 alone (p=0.009) or in addition to ciprofloxacin (p=0.006) or ceftazidime (p=0.004) but not by antibiotics at 2×MIC (p=0.709 ciprofloxacin, p=0.274 ceftazidime).

Minimum Bactericidal Titer

Biofilm elimination was successful at a MOI>0.00001, making phage OMK01 highly effective for the elimination of biofilms. A single treatment of a biofilm with 1,000 PFU was sufficient to remove a 72-hour biofilm containing ˜1⁸ CFU of P. aeruginosa.

Strain Characterization

Resensitization to antibiotics was demonstrated in the strain of P. aeruginosa following evolved resistance to phage OMKO1. The MIC as determined by eTest strip decreased in all four of the examined antibiotics: ciprofloxacin, ceftazidime, gentamicin, and tobramycin. This strain, initially ciprofloxacin resistant (MIC=1.172 mg/L), fell to susceptible levels (MIC=0.014) following resistance to OMKO1. Similar patterns for gentamicin before phage exposure (MIC=3.7 mg/L) and after (MIC=0.833 mg/L) and tobramycin before (MIC=11.33 mg/L) and after (MIC=3.667 mg/L). This strain was initially clinically susceptible to ceftazidime (MIC=1.667 mg/L) but strains with evolved resistance to phage OMKO1 had notably decreased MIC (0.35 mg/L) (FIG. 6).

Case Report Example

Initial pre-procedure imaging appeared to show scarring of the thoracic abscess with little, if any, fluid content. Sampling of the fistular discharge yielded a monoculture of P. aeruginosa as with previous sampling efforts from the same source. The thoracic abscess was accessed through direct needle puncture using image guidance. No fluid could be aspirated back or injected, confirming that the abscess had nearly scarred over. The needle was withdrawn from the chest and 10 mL of phage OMK01 (10⁷ PFU/mL) and ceftazidime (0.2 g/mL) solution was topically applied into the anterior chest fistula. A sterile dressing was placed over the fistula and the patient was admitted to a telemetry monitored bed from where he was discharged with stable vital signs. Approximately 5 weeks after the procedure the patient developed bleeding from an aorto-cutaneous fistula secondary to perforation from ectopic bone. He underwent emergency partial removal of the Dacron graft. By report, cultures at the time of the operative intervention only revealed growth of Candida. The patient has remained off antibiotics for at least nine months with no evidence of recurrent infection.

The ability of a phage to disrupt biofilms where traditional antibiotics fail provides a significant advantage (FIGS. 4A-C) over the old argument for use of phage therapy in biofilm-associated infections and should be considered for difficult-to-eradicate infections. The ability of phage OMK01 to remove 72-hour biofilms containing a clinical isolate from a study patient and on materials relevant to the patient's case was examined. Verification of phage OMK01 efficacy in vitro prior to therapeutic application provided valuable insight into the potential clinical outcome. In this case, there were no noticeable side effects associated with phage OMK01 and it appears to have been effective at biofilm eradication on prosthetic graft material. A case might be made that the partial removal of the infected graft played a role in clearing the infection; however, the demonstrated lack of an abscess on reoperation, the presence of the spicule of ectopic bone visibly causing the fistula, the lack of P. aeruginosa in the operative specimens, and a nine month recurrence free hiatus from the time of initial application, strongly suggest that the phage therapy played a major role in the outcome.

Phage recovered from experimental mice in NIH Preclinical Services study

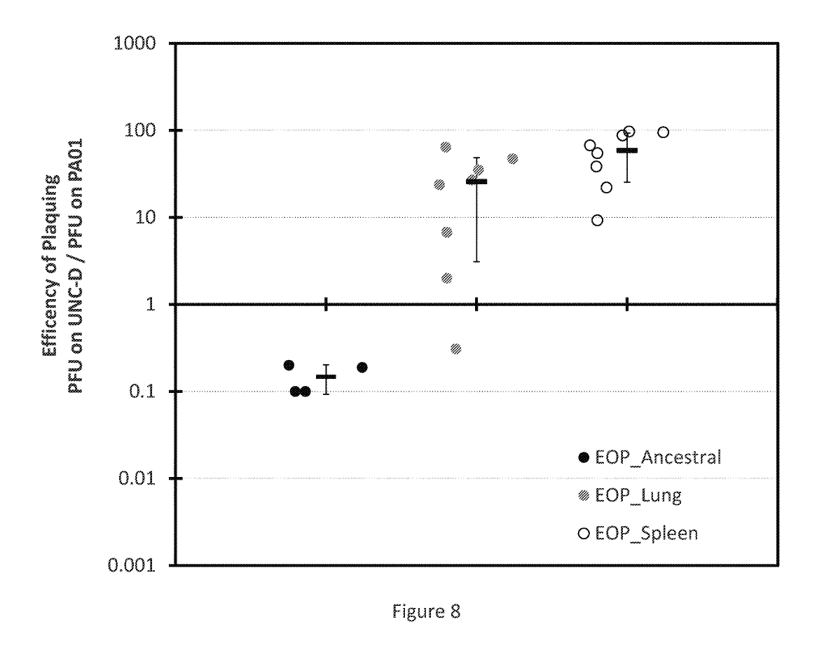

According to results obtained in related studies described herein, results of the murine experiments are expected to illustrate that phage OMKO1 can be used to treat acute pneumonia and resensitize infecting P. aeruginosa strains to chemical antibiotics or disrupt P. aeruginosa, thereby sensitizing P. aeruginosa to one or more antibiotics. Prior to the experiment, it was observed that the ‘ancestral’ phage OMKO1 (i.e., the strain provided for the mouse study) replicated better on P. aeruginosa lab strain PA01, relative to replication on the UNC-D bacterial strain used in the murine model. FIG. 8 shows that the efficiency of plaquing (EOP) for the ancestor phage on UNC-D bacteria is 10-fold less than that observed when the phage was grown on lab strain PA01 (EOP of ˜0.14). In contrast, the vast majority of the phage isolates from the experimental mice replicate better on the pathogenic UNC-D bacteria than on the PA01 bacteria, especially for phages isolated from the spleen samples (FIG. 8). These results indicated that the intrahost phage population rapidly adapted to the infecting UNC-D strain in vivo; only 30 hours of growth on the bacterial pathogen infecting the mouse lung was sufficient for the phage population to greatly improve in targeting these bacteria (mean EOP=25.7 in phage from lung; mean EOP=58.9 in phage from spleen; FIG. 8). From these preliminary data, a conclusion can be reached that phage OMKO1 is generally capable of infecting P. aeruginosa in the murine model of acute lung infection, and that the phage exhibits improved killing efficiency on a target bacterial pathogen. Thus far, results from the murine model further suggest that phage OMKO1 is a strong candidate for development in phage-therapy applications.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of increasing antibiotic sensitivity in pathogenic bacteria, the method comprising:
contacting the bacteria with a lytic bacteriophage, wherein the bacteriophage binds a molecule of an efflux pump in the bacteria and the bacteria either genetically resists bacteriophage infection or becomes infected and lysed by the bacteriophage, and wherein genetically resistant bacteria have impaired efflux pumps and increased sensitivity to antibiotics.

2. The method of claim 1, wherein the bacteria are contacted with bacteriophage at a multiplicity of infection of bacteriophage to bacteria in the range of about 0.05 to about 50.

3. The method of claim 1, wherein the bacteriophage binds a protein of a Mex efflux pump.

4. The method of claim 3, wherein the Mex protein is surface exposed protein.

5. The method of claim 3, wherein the Mex protein is selected from the group consisting of OprM, MexA, MexB, MexX, and MexY.

6. The method of claim 1 further comprising contacting the genetically resistant bacteria with an antibiotic.

7. The method of claim 1, wherein the pathogenic bacteria is a multi-drug resistant (MDR) bacteria.

8. A pharmaceutical composition comprising a lytic bacteriophage, wherein the bacteriophage binds a molecule of an efflux pump on multi-drug resistant (MDR) bacteria.

9. The composition of claim 8 further comprising an antibiotic.

10. A method of treating a multi-drug resistant bacterial infection in a subject in need thereof, the method comprising administering the pharmaceutical composition of claim 8 to the subject with the bacterial infection.

11. The method of claim 10, wherein the pharmaceutical composition is administered directly to a site of the bacterial infection.

12. The method of claim 10 further comprising administering an antibiotic to the subject.

13. The method of claim 12, wherein the antibiotic is administered before or after or co-administered with the pharmaceutical composition.

14. The method of claim 1, wherein the bacteriophage is OMKO1.

15. The method of claim 1, wherein the pathogenic bacteria is associated with a biofilm.

16. The method of claim 1, wherein the pathogenic bacteria is Pseudomonas aeruginosa.

17. The method of claim 16, wherein the Pseudomonas aeruginosa is a Pseudomonas aeruginosa biofilm.

18. The composition of claim 8, wherein the bacteriophage is OMKO1.

19. The composition of claim 8, wherein the bacteria is associated with a biofilm.

20. The composition of claim 8, wherein the bacteria is Pseudomonas aeruginosa.

21. The composition of claim 20, wherein the Pseudomonas aeruginosa is a Pseudomonas aeruginosa biofilm.

22. A method of disrupting a pathogenic bacteria associated with a biofilm, the method comprising:
contacting the bacteria with a lytic bacteriophage, wherein the bacteriophage binds a molecule of an efflux pump in the bacteria and the bacteria either genetically resists bacteriophage infection or becomes infected and lysed by the bacteriophage, and wherein genetically resistant bacteria have impaired efflux pumps and increased sensitivity to one or more antibiotics; and
contacting the genetically resistant bacteria so identified with the one or more antibiotics, thereby disrupting the pathogenic bacteria associated with the biofilm.
